(19) United States
(12) Patent Application Publication
(10) Pub. No.: US 2011/0190585 A1
(43) Pub. Date: Aug. 4, 2011

(54) SURGICAL TOOL, PARTICULARLY FOR USE IN LAPAROSCOPY

(75) Inventors: Achille Lucio Gaspari, Roma (IT); Nicola Di Lorenzo, Roma (IT); Luca Iezzi, Roma (IT)

(73) Assignee: Universita degli Studi di Roma "Tor Vergata"

(21) Appl. No.: 12/992,055

(22) PCT Filed: Apr. 10, 2009
(86) PCT No.: PCT/IB2009/051528

§ 371 Date: Apr. 6, 2011

(30) Foreign Application Priority Data
Apr. 11, 2008 (IT) RM2008A000195

(51) Int. Cl. A61B 1/00

(52) U.S. Cl. 600/118

(57) ABSTRACT

A surgical instrument (3), in particular for use in laparoscopy, comprises means for inducing the surface adsorption of ions in correspondence of said sheath (10) allowing to reducing burns due to electrical dispersions.

The present invention relates to a surgical tool, in particular for use in laparoscopy.

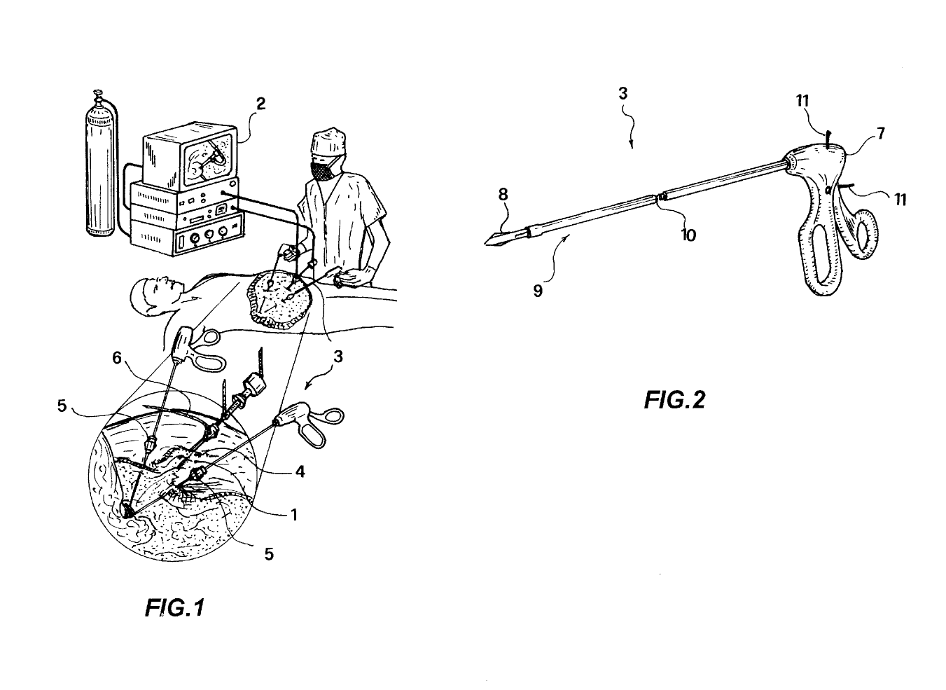

By way of non limiting introduction, a laparoscopic operation is described with reference to FIG. 1, showing an operation of this kind. On the contrary, FIG. 2 shows a surgical instrument or the kind used in laparoscopy.

Laparoscopy substantially consists of a surgical operation in which there no open-cuts are performed in order to show the operating field, in particular a laparotomy, but suitable illuminating instruments and a video camera 1 connected to a monitor 2 are used, together with thin surgical instruments 3, such as tweezers, scissors, diathermocoagulators, surgical staples, needle holders and so on, that are introduced through small holes made in the abdominal wall.

Such arrangements typically require introducing into the abdominal cavity an inert gas, usually carbon dioxide (CO₂), for swelling the abdomen up, in order to provide a sufficient space for moving the instruments.

The video camera 1 is introduced through a suitable incision 4, so as to allow the surgeon to observe the operating field through the monitor 2.

Devices called trocar 5 are also placed through the abdominal wall, allowing to introduce and move the gas intake cannula 6, the video camera 1, the latter possibly integrated with the cannula 6 and the surgical instruments 3, which are operated under visual control.

Surgical instruments 3 have a proximal end 7, having operation and control means of the instrument, and a distal end 8, instead having the surgical instrument.

The diathermocoagulator is an instrument always used, that causes an hemostasis by applying heat obtained from a heating resistor provided at the distal 8, inserted into the abdominal cavity.

Therefore, this device should be connected through appropriate electrical conductors inserted in the central body 9 of the instrument 3, protected by a sheath 10. Electrical contacts 11 for power supplying the instrument 3 are also provided on the proximal end.

Anyhow, it is understood that this is just an example of an instrument wherein the central body is crossed by a current conductor.

The experience of use has shown the likelihood of inflicting burns in patients undergoing laparoscopic operations.

It was noted that such burns, caused by electric current, may be due to:
direct contact: which is determined when energy is transferred through a physical contact between two conductors, this effect being generally attributed to an error by the surgeon;
lack of insulation, attributable to a defect of the instrument apart from the correct control of the surgeon; and
capacitive coupling, which is a mechanism whereby the electric current induced in an electrode produces a current close to nearby conductors, despite the presence of an undamaged insulation between two conductors.

In particular, the capacitive coupling phenomenon occurs with all monopolar electrosurgical instruments but fortunately negative impacts on the patient do not occur often since they depend on how much current is transferred and on its intensity.

It has been noted that this effect increases with increasing tension, in fact in the cutting operation, which uses a lower voltage than the clotting operation, it has a minor injuries incidence.

The present invention relates to this field, aiming to reducing the risk of burns due to electrical dispersion phenomena during surgery performed in laparoscopy.

The above mentioned technical problem is solved by a surgical instrument as above specified, wherein means for inducing the surface adsorption of ions are provided in correspondence of said sheath.

Such means for inducing the surface adsorption of ions comprises, according to an exemplificative embodiment of the present invention, the use of a covering on the sheath, the covering being made of an adsorbent material, allowing the densification of the substance at the separation surface between the solid and liquid phases, on the one hand, and counteracts the effect capacitive, on the other hand.

The chemical and physical phenomena involved are the adsorption and the enhancement of the dielectric strength due to the covering in said material, when used instead of traditional materials.

The chemical process of adsorption consists of a phenomenon according to which, by putting in contact two phases such as solid and liquid, there is a thickening of a substance in one phase on the separation surface between the two phases.

With regard to the dielectric strength, by increasing an electric field between two insulated beyond a limit value, an electric discharge will finally occur, which is avoidable only by increasing the dielectric strength as far as possible.

The provision of adsorption as above specified has the ability to increase the dielectric strength of the sheath 10 due to ion accumulation at the separation between the two phases, that, in fact, insulates what is within said covering.

In order to provide such covering, it is possible to use a polymer as long as it is hypoallergenic, atoxic and not biodegradable.

Among the polymers suitable for providing such covering, preferred polymers are polymethyl-methacrylate, commonly called plexiglass, which has good chemical inertia and is sufficiently adsorbent, and polyolefins, which are extensively used in the field of biomaterials, in the manufacturing of orthopaedic implants where surfaces are subjected to heavy loads, having a good biocompatibility, considerable resistance to repeated bending, excellent resistance to chemicals in the environment and a good permeability to gases.

Among polyolefins, the preferred solution is represented by polytetrafluoroethylene (PTFE or Teflon®).

The main characteristics of polytetrafluoroethylene are:
complete chemical inertia, thanks to which, it is not attacked by almost all chemical composes;
very low dielectric constant (ca. 2.0);
very good electrical properties (dielectric strength equal to about 65 KV/mm);
complete insolubility in water and in any organic solvent;
relatively low cost (about 90 ε/kg);
proved biocompatibility.

Advantageously, the structure of expanded polytetrafluoroethylene, known by the trademarks Impra®, Goretex®, Vitagraft®, can be used, which is achieved by a process of stretching to high temperature, generating PTFE nodules interconnected with highly oriented fibrils.

Polytetrafluoroethylene, in particular in its expanded version, is also preferred for its chemical composition formed by atoms of fluorine and carbon, which makes its matrix particularly porous and are capable to absorb molecules and ions present in biological fluids.

It is intended that the present invention relates in general to reducing the risk of burns both due to electrical insulation defects and to ionic transport phenomena, and to the capacitive effect, in all surgical operations where it is necessary to ensure an electrical insulation in an electrical conductor which is necessarily immersed in organic fluids or wetted by them.

To the above-described surgical instrument, a person skilled in the art, in order to satisfy further and contingent needs, could effect several further modifications and variants, all however encompassed in the protective scope of the present invention, as defined by the appended claims.

1. A surgical instrument, in particular for use in laparoscopy, comprising:
a proximal end, comprising operation and control means of the surgical instrument;
a distal end, adapted to be introduced into an operating field,
a central body, containing electrical conductors shielded by a sheath, and
means for inducing surface adsorption of ions in correspondence of said sheath.

2. The surgical instrument according to claim 1, wherein the means for inducing the surface adsorption of ions comprises a covering made of absorbent material.

3. The surgical instrument according to claim 2, wherein the absorbent material is a polymeric material.

4. The surgical instrument according to claim 3, wherein said absorbent material is selected from a group comprising polythylmethacrylate and polyolefins.

5. The surgical instrument, according to claim 4, wherein said absorbent material is polytetrafluoroethylene.

6. The surgical instrument according to claim 5, wherein the covering is formed by expanded polytetrafluoroethylene.
